(19) United States
(12) Patent Application Publication
(10) Pub. No.: US 2023/0044127 A1
(43) Pub. Date: Feb. 9, 2023

(54) MOLDABLE MEDICAL MEMBRANE

(71) Applicant: NAN YA PLASTICS CORPORATION, TAIPEI (TW)

(72) Inventors: TE-CHAO LIAO, TAIPEI (TW); Min-Fan Chung, Taoyuan City (TW); CHING-YAO YUAN, TAIPEI (TW)

(21) Appl. No.: 17/862,377

(22) Filed: Jul. 11, 2022

(30) Foreign Application Priority Data
Aug. 9, 2021 (TW) 110129214

(51) Int. Cl. A61F 2/28; A61L 27/56; A61C 8/00

(52) U.S. Cl. CPC A61F 2/2846; A61L 27/56; A61F 2/2803; A61C 8/0018; A61F 2002/30062

(57) ABSTRACT

A moldable medical membrane is provided, which includes a compact layer and a porous layer. The compact layer is formed from a first material. The porous layer is disposed on the compact layer, and the porous layer is formed from a second material. The moldable medical membrane has a moldable temperature range. A melting point of the compact layer is within the moldable temperature range, and a melting point of the porous layer is higher than the moldable temperature range.

### CROSS-REFERENCE TO RELATED PATENT APPLICATION

This application claims the benefit of priority to Taiwan Patent Application No. 110129214, filed on Aug. 9, 2021. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a moldable medical membrane, and more particularly to a biodegradable moldable medical membrane.

### BACKGROUND OF THE DISCLOSURE

A guided bone regeneration (GBR) procedure, which is also called a bone repair operation, is usually performed before a dental implant. This can solve problems caused by having a tooth missing for an extended period of time, such as shrinkage of an alveolar bone.

Referring to FIG. 1, during the GBR, a gum tissue G is cut apart, and then bone grafts B are filled in a depression of the alveolar bone, so as to facilitate hyperplasia of bone cells in a tooth ridge R. In a case where a growing space for the bone cells is occupied by the gum tissue G or soft tissues during cell proliferation, a separation membrane F is disposed on the bone grafts B to separate the alveolar bone from the soft tissues. Finally, the gum tissue G is stitched up. Accordingly, the bone cells can grow within a specific space to rebuild the tooth ridge R.

A common separation membrane that is currently available on the market is made from collagen (hereinafter referred to as a collagen membrane). Since physical properties of the collagen membrane are weak, the collagen membrane is likely to rupture after the implant, which causes the artificial bones to fall. Moreover, the collagen membrane is not moldable. In order to completely cover a wound, dentists need to fix the shape of the collagen membrane by sewing or other auxiliary measures. Accordingly, the collagen membrane is inconvenient for use due to its weak physical properties.

Therefore, how to improve the physical properties of the conventional separation membrane and increase its convenience of use, so as to overcome the above-mentioned problems, has become one of the important issues to be addressed in the industry.

### SUMMARY OF THE DISCLOSURE

In response to the above-referenced technical inadequacies, the present disclosure provides a moldable medical membrane.

In one aspect, the present disclosure provides a moldable medical membrane. The moldable medical membrane includes a compact layer and a porous layer. The compact layer is formed from a first material. The porous layer is disposed on the compact layer, and the porous layer is formed from a second material. The moldable medical membrane has a moldable temperature range. A melting point of the compact layer is within the moldable temperature range, and a melting point of the porous layer is higher than the moldable temperature range.

In certain embodiments, when the moldable medical membrane is at a temperature within the moldable temperature range, the compact layer is in a moldable state, and a shape of the porous layer is changed to correspond to a shape of the compact layer.

In certain embodiments, the moldable temperature range ranges from 45° C. to 100° C.

In certain embodiments, a melting point of the first material is lower than 100° C., and a melting point of the second material is not lower than 100° C.

In certain embodiments, a melting point of the first material ranges from 45° C. to 70° C., and a melting point of the second material ranges from 100° C. to 150° C.

In certain embodiments, a viscosity of the first material ranges from 0.20 dl/g to 1.87 dl/g.

In certain embodiments, a viscosity of the second material ranges from 2.00 dl/g to 6.50 dl/g.

In certain embodiments, the porous layer has a porous structure, and a part of the compact layer is disposed in the porous structure.

In certain embodiments, a thickness of the moldable medical membrane ranges from 200 μm to 600 μm.

In certain embodiments, a thickness of the compact layer ranges from 150 μm to 300 μm.

In certain embodiments, a thickness of the porous layer ranges from 50 μm to 400 μm.

In certain embodiments, the first material includes polycaprolactone, and the second material includes polylactic acid.

In certain embodiments, a weight average molecular weight of the first material ranges from 5000 g/mol to 50000 g/mol.

In certain embodiments, a stretching stress of the moldable medical membrane is higher than 10 MPa after the moldable medical membrane is immersed in a saline solution at 37° C. for 30 minutes.

In certain embodiments, a stretching stress of the moldable medical membrane is higher than 10 MPa at a temperature of 25° C. and a relative humidity of 50%.

In certain embodiments, a suture retention strength of the moldable medical membrane is higher than 10 N.

Therefore, in the moldable medical membrane provided by the present disclosure, by virtue of “the melting point of the compact layer being within the moldable temperature range” and “the melting point of the porous layer being higher than the moldable temperature range,” physical properties of the moldable medical membrane can be enhanced, and the moldable medical membrane can have a moldability.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a guided bone regeneration according to the present disclosure; and

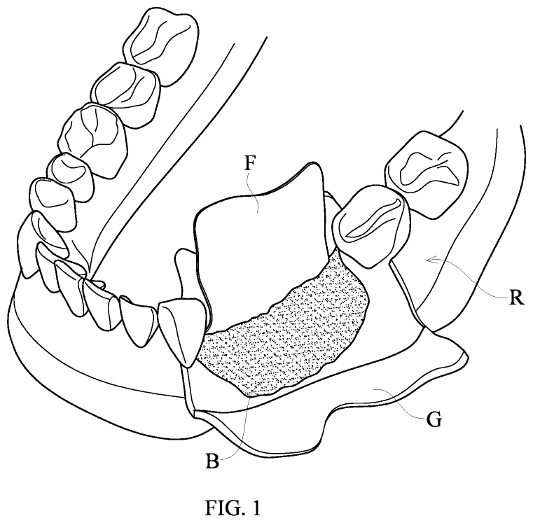

FIG. 2 is a cross-sectional side view of a moldable medical membrane according to the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

A moldable medical membrane of the present disclosure can be used as a separation membrane between bone grafts and soft tissues in a guided bone regeneration. Due to strong physical properties of the moldable medical membrane of the present disclosure, the likelihood of rupture of the separation membrane during a dental implant can be decreased. In addition, the moldable medical membrane of the present disclosure is moldable. Through an appropriate change in temperature, a shape of the moldable medical membrane can be changed so as to be more tightly attached to a wound. Accordingly, the moldable medical membrane of the present disclosure is convenient for use.

In addition to dental surgeries, the moldable medical membrane of the present disclosure can also be used in other surgeries that are related to the human body. However, in order to maintain coherence of description and to express characteristics of the present disclosure in a more detailed manner, dental surgery is taken as an example in the specification.

The moldable medical membrane of the present disclosure has a moldable temperature range. When a temperature of the surrounding environment is within the moldable temperature range, the shape of the moldable medical membrane can be changed. Specifically, the moldable temperature range can range from 45° C. to 100° C. It should be noted that the moldable temperature range of the present disclosure includes any temperature range from 45° C. to 100° C. (45° C. and 100° C. included). In other words, an upper limit and a lower limit can be any integers ranging from 45° C. to 100° C., such as, but not limited to, 45° C., 50° C., 55° C., 60° C., 65° C., 70° C., 75° C., 80° C., 85° C., 90° C., 95° C., or 100° C.

When the moldable medical membrane of the present disclosure is used as a separation membrane, the shape of the moldable medical membrane can be changed to correspond to a shape of an alveolar bone by immersing the moldable medical membrane into a deionized water or placing the moldable medical membrane onto a heating plate at a temperature that ranges from 45° C. to 100° C. After being cooled, the moldable medical membrane is reshaped. The reshaped moldable medical membrane can be disposed on the alveolar bone and the bone grafts, so as to completely cover the bone grafts and separate the bone grafts from the soft tissues. Accordingly, an effect of the guided bone regeneration can be enhanced.

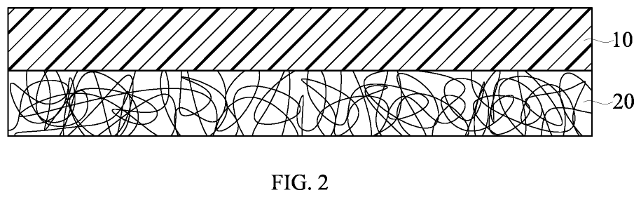

Referring to FIG. 2, the moldable medical membrane of the present disclosure includes a compact layer 10 and a porous layer 20. The porous layer 20 is disposed on the compact layer 10.

The compact layer 10 has strong physical properties, which can enhance the physical properties of the overall moldable medical membrane. Pores are absent from the compact layer 10. As such, epithelial cells and the bone cells can be effectively isolated, and a growing space for the bone cells will not be occupied by the epithelial cells during cell proliferation. In addition, the compact layer 10 has a moldability, and a melting point of the compact layer 10 is within the moldable temperature range. Accordingly, when the compact layer 10 is at a temperature within the moldable temperature range, the compact layer 10 is in a moldable state, and a shape of the compact layer 10 can be changed.

A density of the porous layer 20 is lower than a density of the compact layer 10. The porous layer 20 has a porous structure, and the porous layer 20 is flexible. In an exemplary embodiment, a part of the compact layer 10 is formed in the porous structure of the porous layer 20, such that the compact layer 10 and the porous layer 20 can be tightly combined. When the moldable medical membrane is used, the porous layer 20 contacts the alveolar bone, such that periodontal tissues can attach to and grow on the porous structure of the porous layer 20, so as to promote regeneration and mending of the alveolar bone. In addition, a melting point of the porous layer 20 is higher than the moldable temperature range. When the moldable medical membrane is at a temperature within the moldable temperature range, the porous layer 20 can support the compact layer 10 when not in a moldable state. Due to flexibility of the porous layer 20, a shape of the porous layer 20 can be changed corresponding to the shape of the compact layer 10. Accordingly, the moldable medical membrane of the present disclosure has not only moldability but also strong physical properties.

The compact layer 10 is formed from a first material. The porous layer 20 is formed from a second material. The first material is different from the second material. By using two different materials, the moldable medical membrane of the present disclosure has strong physical properties at room temperature, and has moldability at a temperature within the moldable temperature range (from 45° C. to 100° C.).

Specifically, the melting point of the first material is lower than 100° C., and the melting point of the second material is higher than 100° C. Further, the melting point of the first material ranges from 45° C. to 70° C., and the melting point of the second material ranges from 100° C. to 150° C. Accordingly, the compact layer 10 contributes to the moldability of the moldable medical membrane.

The compact layer 10 is in a moldable state at a temperature within the moldable temperature range, and the shape of the moldable medical membrane can be changed. Given that the melting point of the porous layer 20 is higher than the moldable temperature range, a structure of the porous layer 20 can be maintained during a molding process, so as to support the compact layer 10. In addition, the shape of the porous layer 20 is changed corresponding to the shape of the compact layer 10, and a deformation stress is stored in the porous layer 20 due to deformation. Since a temperature change will not cause damage to the porous structure of the porous layer 20, the porous layer 20 remains flexible.

After the moldable medical membrane is cooled to room temperature, the shape of the compact layer 10 is fixed, and the shape of the porous layer 20 is the same as that of the compact layer 10. Accordingly, the shape of the overall moldable medical membrane of the present disclosure is moldable.

On the other hand, a viscosity of the first material ranges from 0.20 dl/g to 1.87 dl/g. A viscosity of the second material ranges from 2.00 dl/g to 6.50 dl/g. The porous layer 20 contributes to the strong physical properties of the moldable medical membrane, thereby solving the problem of conventional separation membranes being prone to rupture.

In order to enhance the convenience of use, a thickness of the moldable medical membrane of the present disclosure is adjusted to range from 200 μm to 600 μm. In order to balance the moldability and the physical properties, a thickness of the compact layer 10 is thinner than or equal to a thickness of the porous layer 20. In an exemplary embodiment, the thickness of the compact layer 10 ranges from 150 μm to 300 μm. For example, the thickness of the compact layer 10 can be 175 μm, 200 μm, 225 μm, 250 μm, or 275 μm. The thickness of the porous layer 20 ranges from 50 μm to 400 μm. For example, the thickness of the porous layer 20 can be 75 μm, 100 μm, 125 μm, 150 μm, 175 μm, 200 μm, 225 μm, 250 μm, 275 μm, 300 μm, 325 μm, 350 μm, or 375 μm.

In some embodiments, adjusting properties of the first material can also achieve an effect of upholding the moldablility and the strong physical properties of the moldable medical membrane of the present disclosure. The first material can be a biodegradable polymer having a weight average molecular weight ranging from 5000 g/mol to 50000 g/mol, so as to enable the compact layer 10 to be molded at a temperature within the moldable temperature range. Preferably, the first material is a biodegradable polymer having a weight average molecular weight ranging from 10000 g/mol to 30000 g/mol. In an exemplary embodiment, the first material includes polycaprolactone (PCL). However, the aforementioned examples describe only one of the embodiments of the present disclosure, and the present disclosure is not intended to be limited thereto.

The second material can be another biodegradable polymer. The second material can be a biodegradable polymer having a weight average molecular weight ranging from 100000 g/mol to 600000 g/mol. Preferably, the second material is a biodegradable polymer having a weight average molecular weight ranging from 150000 g/mol to 350000 g/mol. In an exemplary embodiment, the second material includes polylactic acid (PLA). However, the aforementioned examples describe only one of the embodiments of the present disclosure, and the present disclosure is not intended to be limited thereto.

The moldable medical membrane of the present disclosure can be manufactured by a method below, but is not limited thereto. The porous layer 20 can be formed by a non-woven spinning technology, a freeze-drying technology, or an electrostatic spinning technology.

Taking the electrostatic spinning technology as an example, a polymer solution for forming the porous layer 20 needs to be prepared first. The polymer solution is non-toxic (or low toxic). The polymer solution includes a polymer material and a solvent. An amount of the polymer material in the polymer solution ranges from 1 wt % to 50 wt %, and an amount of the solvent in the polymer solution ranges from 50 wt % to 99 wt %.

The polymer material can be selected from the group consisting of polylactic acid, polycaprolactone, polyhydroxyalkanoate (PHA), and polyglycolic acid (PGA). The solvent can be selected from the group consisting of acetone, methyl ethyl ketone, ethylene glycol, isopropanol, deacetylated chitin (DAC), N, N-dimethylformamide (DMF), dimethylacetamide (DMAC), dimethyl methylene (DMSO), and ether.

In an exemplary embodiment, the polymer material is polylactic acid. When stability and quality of electrospinning are taken into consideration, the solvent is a mixture of acetone and dimethylacetamide. A weight ratio of acetone to dimethylacetamide can range from 1:9 to 9:1.

The prepared polymer solution is added into a storage tank. A nozzle and a collector are respectively and electrically connected to a positive electrode and a negative electrode of a high-voltage power supply. After the high-voltage power supply is switched on, the polymer solution is sprayed from the nozzle. Due to an electric field generated by the high-voltage power supply, the polymer solution is solidified to form polymer fibers. Then, the polymer fibers are deposited on the collector. Through adjusting a movement of the nozzle, the polymer fibers can be tightly stacked, entangled, or interwoven along a specific direction, so as to form the porous layer 20 with a uniform thickness.

In an exemplary embodiment, a temperature of the polymer solution can range from 5° C. to 95° C. Preferably, the temperature of the polymer solution ranges from 10° C. to 90° C. A voltage set on the high-voltage power supply ranges from 5 KV to 60 KV. Preferably, the voltage set on the high-voltage power supply ranges from 10 KV to 25 KV. A feed rate of the polymer solution can range from 0.1 cc/min to 5 cc/min. A distance between a spinning tip of the nozzle to the collector ranges from 15 cm to 90 cm.

After the porous layer 20 is formed, the compact layer 10 can be disposed on the porous layer 20 by thermopressing, so as to obtain the moldable medical membrane of the present disclosure. When the compact layer 10 is disposed on the porous layer 20 by thermopressing, a part of the compact layer 10 permeates into the porous structure. In this way, the compact layer 10 and the porous layer 20 can be tightly combined.

To prove that the moldable medical membrane of the present disclosure can overcome the inadequacies of the conventional separation membrane (i.e., weak physical properties and lack of moldability), the moldable medical membrane of the present disclosure is manufactured by the method mentioned previously. Then, stretching stresses and suture retention strengths of the moldable medical membrane of the present disclosure and the conventional separation membranes are measured for comparison. Specific results of the stretching stresses and the suture retention strengths are listed in Table 1.

In Table 1, the stretching stress (dry) refers to a stretching stress of the moldable medical membrane/the conventional separation membrane that is tested at a temperature of 25° C. and a relative humidity of 50%. The stretching stress (wet) refers to a stretching stress of the moldable medical membrane/the conventional separation membrane that is tested after being immersed into a saline solution at 37° C. for 30 minutes. The suture retention strength is tested according to the standard of ISO 7198.

TABLE 1

| | Moldable medical | | | | | | | | | | | |
|---|---|---|---|---|---|---|---|---|---|---|---|---|
| | membrane of the | | | | | | | | | | | |
| | present disclosure | | Conventional separation membrane | | | | | | | | | |
| Brand (model) | — | | Zimmer Biomet | | Nano Sigma | | Geistlich | | Ossix (OSSIX | | Curasan | |
| | | | (OS SEOGUARD ®) | | Biotech | | (BIO-GIDE ®) | | PLUS ®) | | (EPIGUIDE ®) | |
| Material | PCL/PLA | | Crosslinked collagen | | Uncrosslinked collagen | | | | | | PLA | |
| Thickness | 400 | μm | 240 | μm | 100 | μm | 400 | μm | 260 | μm | 388 | μm |
| Stretching stress | 14.2 | MPa | 29.1 | MPa | 2.18 | MPa | 4.4 | MPa | 5.3 | MPa | 0.6 | MPa |
| (dry) | | | | | | | | | | | | |
| Stretching stress | 10.6 | MPa | 5.7 | MPa | 1.5 | MPa | 3.4 | MPa | 2.9 | MPa | 0.5 | MPa |

| (wet) | | | | | | | | | |
|---|---|---|---|---|---|---|---|---|---|
| suture retention | 11.262 | N | 5.769 | N | 0.824 | N | — | — | — |

strength

According to Table 1, the moldable medical membrane of the present disclosure has good stretching stresses in both of a dry environment and a wet environment. Even in the wet environment, the moldable medical membrane of the present disclosure can still have appropriate stretching stresses.

In comparison, the stretching stresses of the conventional separation membranes are weak in the wet environment. The conventional separation membrane made from crosslinked collagen has a strong stretching stress in the dry environment. However, once the environment becomes wet, the stretching stress of the conventional separation membrane made from the crosslinked collagen is dramatically decreased. Therefore, the conventional separation membranes made from the crosslinked collagen still have the problem of weak physical properties at certain conditions. In addition, the conventional separation membranes made from uncrosslinked collagen or polylactic acid have weak physical properties whether in the dry environment or the wet environment.

Specifically, the stretching stress of the moldable medical membrane of the present disclosure after being immersed in the saline solution at 37° C. for 30 minutes (i.e., the stretching stress (wet)) is higher than 10 MPa. The stretching stress of the moldable medical membrane of the present disclosure at the temperature of 25° C. and the relative humidity of 50% (i.e., the stretching stress (dry)) is higher than 10 MPa.

Moreover, the moldable medical membrane of the present disclosure has a strong suture retention strength. Therefore, in practical application, the moldable medical membrane of the present disclosure is less likely to rupture and is convenient for use.

Specifically, according to the standard of ISO 7198, the suture retention strength of the moldable medical membrane of the present disclosure is higher than 5N. Preferably, the suture retention strength of the moldable medical membrane of the present disclosure is higher than 8N. More preferably, the suture retention strength of the moldable medical membrane of the present disclosure is higher than 10N.

Therefore, while the physical properties (the stretching stress and the suture retention strength) of the conventional separation membranes are poor, the moldable medical membrane of the present disclosure can overcome these inadequacies. In addition, the moldable medical membrane of the present disclosure further has moldability. Accordingly, compared to the conventional separation membranes, the moldable medical membrane of the present disclosure is more convenient for use.

### Beneficial Effects of the Embodiments

In conclusion, in the moldable medical membrane provided by the present disclosure, by virtue of “the melting point of the compact layer 10 being within the moldable temperature range” and “the melting point of the porous layer 20 being higher than the moldable temperature range,” the physical properties of the moldable medical membrane can be enhanced, and the moldable medical membrane can have moldability.

Further, by virtue of “the melting point of the first material being lower than 100° C., and the melting point of the second material being higher than 100° C.”, the moldable medical membrane of the present disclosure is moldable.

Further, by virtue of “the viscosity of the first material ranging from 0.20 dl/g to 1.87 dl/g, and the viscosity of the second material ranging from 2.00 dl/g to 6.50 dl/g”, the moldable medical membrane of the present disclosure can have the strong physical properties and moldability.

Further, by virtue of “the porous layer 20 having the porous structure, and a part of the compact layer 10 being disposed in the porous structure”, the physical properties of the moldable medical membrane of the present disclosure can be improved.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A moldable medical membrane, comprising:
a compact layer formed from a first material; and
a porous layer disposed on the compact layer, the porous layer being formed from a second material;
wherein the moldable medical membrane has a moldable temperature range, a melting point of the compact layer is within the moldable temperature range, and a melting point of the porous layer is higher than the moldable temperature range.

2. The moldable medical membrane according to claim 1, wherein, when the moldable medical membrane is at a temperature within the moldable temperature range, the compact layer is in a moldable state, and a shape of the porous layer is changed to correspond to a shape of the compact layer.

3. The moldable medical membrane according to claim 1, wherein the moldable temperature range ranges from 45° C. to 100° C.

4. The moldable medical membrane according to claim 1, wherein a melting point of the first material is lower than 100° C., and a melting point of the second material is not lower than 100° C.

5. The moldable medical membrane according to claim 1, wherein a melting point of the first material ranges from 45° C. to 70° C., and a melting point of the second material ranges from 100° C. to 150° C.

6. The moldable medical membrane according to claim 1, wherein a viscosity of the first material ranges from 0.20 dl/g to 1.87 dl/g.

7. The moldable medical membrane according to claim 1, wherein a viscosity of the second material ranges from 2.00 dl/g to 6.50 dl/g.

8. The moldable medical membrane according to claim 1, wherein the porous layer has a porous structure, and a part of the compact layer is disposed in the porous structure.

9. The moldable medical membrane according to claim 1, wherein a thickness of the moldable medical membrane ranges from 200 μm to 600 μm.

10. The moldable medical membrane according to claim 1, wherein a thickness of the compact layer ranges from 150 μm to 300 μm.

11. The moldable medical membrane according to claim 1, wherein a thickness of the porous layer ranges from 50 μm to 400 μm.

12. The moldable medical membrane according to claim 1, wherein the first material includes polycaprolactone, and the second material includes polylactic acid.

13. The moldable medical membrane according to claim 1, wherein a weight average molecular weight of the first material ranges from 5000 g/mol to 50000 g/mol.

14. The moldable medical membrane according to claim 1, wherein a stretching stress of the moldable medical membrane is higher than 10 MPa after the moldable medical membrane is immersed in a saline solution at 37° C. for 30 minutes.

15. The moldable medical membrane according to claim 1, wherein a stretching stress of the moldable medical membrane is higher than 10 MPa at a temperature of 25° C. and a relative humidity of 50%.

16. The moldable medical membrane according to claim 1, wherein a suture retention strength of the moldable medical membrane is higher than 10 N.
